(19) United States
(12) Patent Application Publication
(10) Pub. No.: US 2015/0290129 A1
(43) Pub. Date: Oct. 15, 2015

(54) METHOD FOR DELIVERY OF IMMUNOMODULATORS TO A PATIENT

(71) Applicant: ROCA Medical Ltd., London (GB)

(72) Inventors: James Strader, Austin, TX (US); Jovan Hutton Pulitzer, Frisco, TX (US)

(21) Appl. No.: 14/685,573

(22) Filed: Apr. 13, 2015

Related U.S. Application Data
(60) Provisional application No. 61/978,420, filed on Apr. 11, 2014.

(51) Int. Cl. A61K 9/06; A61K 39/35; A61K 9/00; A61K 39/00

(52) U.S. Cl. CPC A61K 9/06; A61K 39/00; A61K 39/35; A61K 9/0014

(57) ABSTRACT

A method for delivering an immunomodulator to a patient includes providing a bottle of concentrated immunomodulator extract; progressively diluting the antigen extract in sterile bottles; selecting a prescribed amount from a desired one of the dilution bottles; providing a viscous encapsulation material that is able to introduce antigens contained therein through the skin of a patient; introducing one or more doses of the selected prescribed amount of diluted immunomodulator into the viscous encapsulation material; disposing a prescribed amount of viscous encapsulation material containing the introduced diluted immunomodulator therein within a container that is able to dispense such viscous encapsulation material containing the introduced diluted immunomodulator; dispensing from the container the amount of viscous encapsulation material containing the diluted immunomodulator in an amount equal to a single dose; and applying the dispensed viscous encapsulation material containing the introduced diluted immunomodulator to the skin by the patient or a medical professional.

### CROSS-REFERENCE TO RELATED APPLICATIONS

This application claims the benefit of U.S. Provisional Application No. 61/978,420, filed Apr. 11, 2014, entitled STANDARDIZING THE PROCESS FOR CREATING ALLERGY IMMUNOTHERAPY TREATMENTS FOR PATIENTS USING TRANS-DELIVERY DERMAL METHODS AND UTILIZING A CREAM FOR THE LOCAL TARGET DELIVERY OF ANTIGENS FOR TREATMENT OF ALLERGIES, the specification of which is incorporated herein it its entirety.

### TECHNICAL FIELD

That this application is generally related to the delivery of immunomodulators to a patient and, more particularly, to the use of carriers for the dispensing of such across the dermis of the patient.

### BACKGROUND

Immunotherapy (IT) is recognized as most curative treatment for allergies. By exposing the immune system to slowly increasing concentrations of immunomodulators such as an allergen or antigen, it will eventually stabilize and regain control the portion that is hypersensitive to the allergen or antigen. In general, immunotherapy is the “treatment of disease by inducing, enhancing, or suppressing an immune response.” Immunotherapies designed to elicit or amplify an immune response are classified as activation immunotherapies, while immunotherapies that reduce or suppress are classified as suppression immunotherapies. The active agents of immunotherapy are collectively called immunomodulators. They are a diverse array of recombinant, synthetic and natural preparations, often cytokines.

Immunotherapy involved in the treatment of allergies is a type of suppression immunotherapy, often termed desensitization or hypo-sensitization. This is compared with allergy treatments such as antihistamines or corticosteroids which treat only the symptoms of allergic disease. Immunotherapy is the only available treatment that can modify the natural course of the allergic is, by reducing sensitivity to the immunomodulators such as antigens or allergens. An antigen and an allergen and both cause one's immune system to respond. An allergen is an antigen, but not all antigens are allergens. An antigen is any substance that is capable of causing one's immune system to produce antibodies. They are typically organic, or living, produced proteins. An allergen is any antigen that causes an allergic reaction. A non-allergen antigen could be a bacteria, virus, parasite, or fungus that causes an infection. This could also be something else that causes antibody immune system response, like toxins, chemicals, tissue cells involved in transplants or blood cells from a blood transfusion. An allergen is an environmentally produced substance that causes an allergic reaction, although the substance may not be harmful. Allergens cause no reactions in some individuals, while possibly causing a hypersensitive reaction in others. Common allergens include such things as pollen, plants, smoke, feathers, perfumes, dust mites, toxic mold, food, drugs, animal dander, and insect bites and stings.

The exact mechanisms of how IT works are not fully understood, but they involve shifting a patient's immune response from a predominantly “allergic” T-lymphocyte response to a “non-allergic” T-lymphocyte response.

Current accepted processes for performing allergy immunotherapy include injecting immunomodulators matter in the form of antigen material into patient subjects. This is referred to as subcutaneous immunotherapy (SCIT), requiring a patient to visit a doctor's office for weekly injections. It's is very expensive and time-consuming. A second technique, Sublingual immunotherapy (SLIT), involves the application of allergy extracts (antigens), and allergens placed into a pill form and swallowed by the patient or disposed in “allergy drops” which are placed under the tongue for the allergens/antigens to be absorbed into the oral mucosa. Transdermal patches may have been used without much success and mostly were used for patch testing to see if a patient reacts to various chemicals or allergens.

Of the people who start traditional subcutaneous injected immunotherapy (SCIT), 90% fail to complete their therapy due to needle fatigue and not being able to see a doctor in their office once or more per week for several years. Further, doctors charge for every one of those visits. Further, doctors trained to give injections for allergy are concentrated in high population and upper middle class places. People in rural areas and people who do not live in upper middle class areas cannot get to an allergist for shots. Consider an inner city kid having to ride public transportation and pay a high copay just to get a high risk injection if an alternative therapy were available?!

Allergies are also linked to depression and suicide and are among the top ten reasons for missed work and lost productivity. Lastly, allergies and asthma result in billions of dollars in lost productivity and healthcare costs among the 90% of allergy patients that either never get immunotherapy or fail immunotherapy delivered under its current administration methods.

### SUMMARY

In one aspect thereof, a method for delivering an immunomodulator to a patient includes providing a bottle of concentrated immunomodulator extract; progressively diluting the antigen immunomodulator in sterile bottles approved for such dilution; selecting a prescribed amount from a desired one of the dilution bottles at the progressive dilution level required to provide a desired therapeutic effect to a patient as a dose; providing a viscous encapsulation material that is able to transdermally introduce antigens contained therein through the skin of a patient; introducing one or more doses of the selected prescribed amount of diluted immunomodulator into the viscous encapsulation material; disposing a prescribed amount of viscous encapsulation material containing the introduced diluted immunomodulator therein within a container that is able to dispense such viscous encapsulation material containing the introduced diluted immunomodulator in at least a discrete amount associated with each dose contained therein; dispensing from the container the amount of viscous encapsulation material containing the introduced diluted immunomodulator in an amount equal to a single dose; and applying the dispensed amount of viscous encapsulation material containing the introduced diluted immunomodulator to the skin by the patient or a medical professional.

### BRIEF DESCRIPTION OF THE DRAWINGS

For a more complete understanding, reference is now made to the following description taken in conjunction with the accompanying Drawings in which:

FIG. 1 illustrates a diagrammatic view of a dilution sequence of diluting a concentrated antigen extract;

FIG. 2 illustrates step of mixing a dose of the diluted antigen into a container of transdermal cream;

FIG. 3 illustrates a delivery device for delivering specified doses;

FIG. 4 illustrates a process flow for diluting an antigen extract;

FIG. 5 illustrates a process flow for mixing the diluted antigen with a transdermal cream;

FIG. 6 illustrates a process flow of the application of antigen via the transdermal cream;

FIG. 7 illustrates a process flow for the overall distribution chain;

FIG. 8 illustrates a process flow for multiple extracts; and

FIG. 9 illustrates an alternate embodiment of FIG. 8.

### DETAILED DESCRIPTION

Referring now to the drawings, wherein like reference numbers are used herein to designate like elements throughout, the various views and embodiments of a method for delivering immunomodulators to a patient for the treatment of allergies across the skin.are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

The principles of the present disclosed embodiment include a process that involves the use of transdermal carrier creams infused with antigens that can be applied to the patient's skin surface. Further, the antigens can also be carried across the dermal layer along with antihistamines, anti-inflammatory medications, steroids as well as other drugs that may prevent a severe allergic reaction in patients and thus possibly avoid life-threatening anaphylactic reactions. The antigens can also be carried to various depths based upon the physician's requirements. Some transdermal carrier creams penetrate just beyond the deepest skin layer where many of the human cells that recognize antigens as foreign invaders reside. If the physician wishes to treat the patient in a more traditional manner, some transdermal carrier creams can penetrate the patient well below the skin and reach areas of high vascularity thus pushing the antigens into the blood stream much the same as utilizing a hypodermic needle.

The transdermal carrier creams can be used to carry only antigens into the body or they can be used in conjunction with antigens and other medications that can be carried across the skin as combination therapies for allergies.

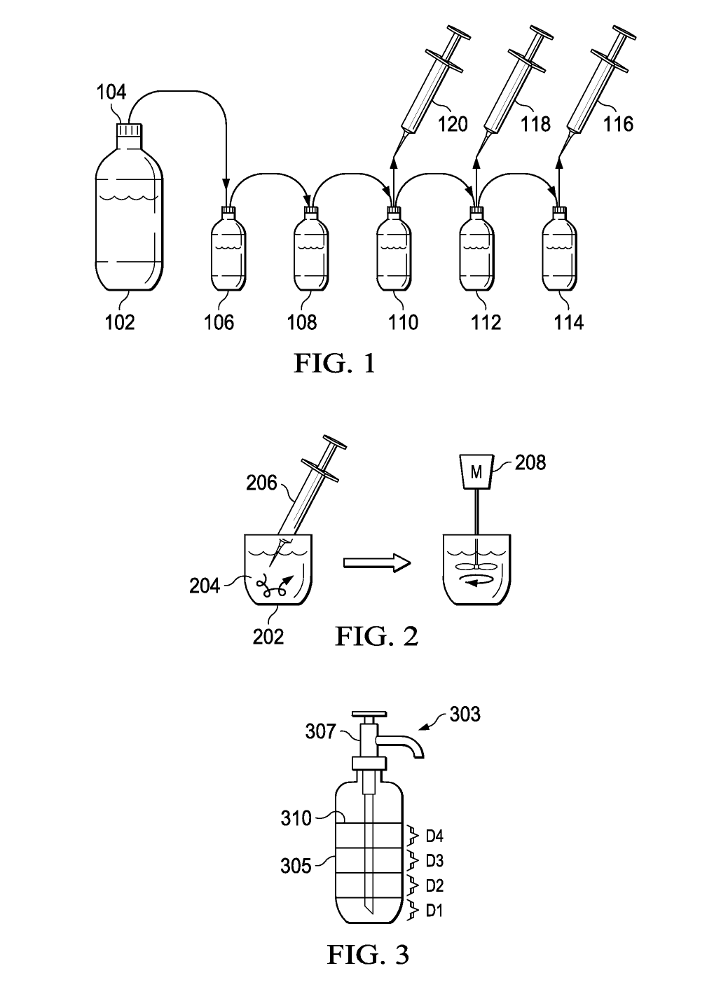

Referring now to FIG. 1, there is illustrated a depiction of a technique for diluting immunomodulators such as antigens, as one example. Preparation of a diluted antigen is performed first by receiving a bottle of extract concentrates from an approved vendor. These are formulated in a given weight/volume (w/v) format with a given antigen associated therewith. For typical antigens such as those associated with the cat antigen, these are relatively well controlled. Typically, a vendor will provide an extract for a single antigen or allergen. Allergens such as pollen and the such are not as well controlled due to the technique for collecting such. In any event, there are typically very few approved vendors for these extracts and allergist typically receives these vendor provided concentrates in a sufficient quantity to make the necessary diluted solution.

Allergen extract is typically comprised of a non-allergenic material, a non-allergenic protein and an allergenic protein. The extraction solutions can be aqueous containing saline and phenol work could be a glycerinated solution. The allergen is added, the units of measure are sometimes referred to as “AU” for “ allergy units,” typically used for mites. These are referred to as “AU/mL.” For such things as grass and cat, the term “BAU” is used for “bioequivalent units.” For other allergens, the terminology is, for example, 1:20 w/v, which stands for 1 g source material per 20 mL of fluid. The relationship between BAU and 1:20 w/v depends upon the extract. In any event, there is a defined amount of extract contained within the concentrate.

When concentrated extracts are formulated by an authorized vendor, they are typically provided in standardized versions and non-standardized versions. In standardized versions, they typically are provided in a 50% glycerin dilutant. They can either be a single allergen extract or they can be a mix. For example, one can obtain a “9 Southern Grass Mix (concentrate)” which contains equal parts of: 2 Bermuda at 10,000 BAU/mL, P27 7 Grass at 100,000 BAU/mL, 15 Johnson at 1:20 w/v. For non-standardized extracts, these are typically provided in either a glycerin dilutant or an aqueous dilutant such as saline. They can be a single extract or a mix. Thus, whenever a concentrated extract is referred to hereinbelow, this refers to a formulation that is provided by an authorized vendor that can be diluted in accordance with the processes described hereinbelow. These are typically provided in the 50 mL bottles with a needle compatible.

Referring back to FIG. 1, the extract concentrate is disposed in a bottle 102. This is a sterile concentrate that has an injection stoppered top 104. There are provided a plurality of five 5 mL sterile injection stoppered bottles 106, 108, 110, 112 and 114, although there could be more and the bottles or containers could be larger than 5 mL. Each of these bottles has disposed therein a defined amount of dilutant, depending upon what the final required to be. Typically, the amount of dilutant is 4.5 mL. The procedure is to, first, extract a defined amount of the concentrated extract from the bottle 102 and dispose it in the bottle 106. This is facilitated by the sterile hypodermic that is inserted through the stopper at the top of the bottle 102 to extract concentrate and then the hypodermic is inserted to the stopper in the bottle 106 to inject extract from bottle 102 into bottle 106. Typically, the concentration in the concentrated extract bottle 102 is 1:20 w/v. This will result in a dilution of 1:10 in bottle 106. If the amount injected is 0.45 mL. Then, 0.45 mL of the diluted solution from bottle 106 is then extracted and inserted into bottle 108, resulting in a 1:100 dilution of the original concentrate in model 108. The process is repeated up to the bottle 114 to provide a solution that is at a dilution of 1:100,000 of the original concentrate. This is a conventional way to provide a selected dilution of the original antigen. However, it should be understood that any concentration level can be provided from one bottle to the next. Purpose of using the sequential bottles is to allow an achievable portion of one bottle to be distributed to the next bottle, rather than trying to extract a very small amount of the initial concentrated extract. Typically, an allergist will then extract from the desired dilution an amount of the diluted antigen for injection percutaneously. Typically, desensitization is achieved by using the most diluted antigen level initially and sequentially moving up to a higher concentration level over time 1.

Illustrated in FIG. 1 are three hypodermic needles, one selecting a “dose” from bottle 114, and labeled hypodermic 116, a second hypodermic needle 118 for retrieving a dose from bottle 112, a third hypodermic needle 120 for extracting a dose from bottle 110. Each of the hypodermic needles 116, 118 and 120 will contain a different diluted dose. These would typically be separate needles in the event that the allergist or medical professional is injecting a patient. For other purposes, they could be the same needle, depending upon the dose or concentration required. An “dose” is defined the amount all the diluted product that would be required for the desired immunotherapy. This is defined by the medical professional. If, for example, bottle 112 were utilized, it may be that 1 mL of diluted solution constituted a “dose.” It could be that less than 1 mL constituted an “dose.”

Referring now to FIG. 2, there is illustrated a diagrammatic view of injecting a dose into a container 202 which contains a quantity of transdermal cream 204, which transdermal cream is a viscous encapsulation material for containing this dose in an emulsion. A hypodermic needle 206 is illustrated which contains a single dose or multiple doses as noted hereinabove, the last bottle 114 will have 5 mL of diluted antigen disposed therein. If the allergist determines that a dose is 1 mL, then a single dose could be disposed in the quantity of transdermal cream 204. If the allergist or medical professional determined that the container 202 should contain three doses, 3 mL would be selected and disposed within the container 202. It is noted that one limitation of the bottle 114 for the 1:100,000 dilution level or the bottle 112 for the 1:10,000 dilution level each has a finite volume. Since the finite volume is, in this example, 5 mL, and if the dose is determined to be 1 mL, then only 5 doses could be provided for a getting a given dilution process. Thus, only a maximum of five doses could be disposed within the container 202. Once the diluted solution of the immunomodulators in the form of an antigen is injected into the transdermal cream 204, it is subjected to a mixing operation via a mixer 208.

A transdermal cream is basically a viscous encapsulation material that includes a base that is provided to transport drugs through the skin. There are some types of transdermal bases or creams that allow for delivery of up to four drugs or compounds through the skin simultaneously. The creams are different than transdermal patches in that they are rubbed on a particular area of the skin and are absorbed through the skin in a very short period of time and are well suited to small molecule antigens. One such transdermal cream is that manufactured by PCCA under the trademark Lipoderm®. This base is utilized for the percutaneous absorption of drugs through the skin. As such, not only can antigens be provided in a particular dose within the transdermal cream, but other drugs such as antihistamines and pain medications can also be provided, depending upon the particular needs of a patient. The desire for the immunomodulators in the form of antigens is that they be disposed within the subcutaneous region just beneath skin or the stratum corneum, which is the outermost layer of the epidermis. This layer, of course, very thin thickness throughout the body. The desire is to allow, via the transdermal cream, the transport of the immunomodulators through this stratum corneum to the subcutaneous tissue or the hypodermis.

Referring now to FIG. 3, there is illustrated a diagrammatic drawing of a distribution pump 303 that has a container 305 and a pump head 307 associated therewith. Disposed within the interior of the container 305 is a quantity of transdermal cream that represents four doses of the antigen at the desired dilutant level. Thus, for example, the last bottle 114, as depicted in FIG. 1, would have around 5 mL of diluted antigen disposed therein. If the desired dosage for the patient was 1 mL, then 4 mL would be extracted from the bottle 114 to provide 4 doses and disposed within a sufficient amount of transdermal cream to rise to a level 310 within the container 305. The dosage increment is that represented by at least one full pump stroke or a defined number of pump strokes. For example, the directions might state that a single full pump stroke represented a single dose and that was what was to be applied at a particular time in accordance with the directions of a medical professional. It might be that multiple stroke are required per dose. In that case, the directions would indicate that the dose is to be applied would require that number of pump strokes. All that would be required would be to know the amount of transdermal cream that was required to fill the container 305 to the level 310 and then inject for dosages therein and mix the material. The transdermal cream could be a transdermal cream that already had a predetermined amount of other medications contained therein such as antihistamines and/or pain medication.

In addition, a sealed straw type tube could be used instead of the metered pump. In this type of delivery device, a premeasured amount of the viscous material, i.e., the transdermal cream, can be provided that could represent, for example, a single dose. However, as described hereinbelow, the amount of cream for this single dose represents a volume of cream that contains, in one example, a single diluted dose of a single concentrated extract or, alternatively, a single dose each of multiple concentrated extracts. For the latter, all that is required is that the volume of the transdermal cream first be defined and then the single dose of each of the concentrated extracts disposed therein.

The transdermal cream or carrier is, as described above, a viscous encapsulation material for encapsulating a finite amount of immunomodulators or antigen therein. This transdermal cream is basically a consolidator. As such, more than a single immunomodulator can be disposed within the cream of This, of course, depends upon what the smallest increment of dosage is comprised of If, for example, the container 202 in FIG. 2 contained 30 g of encapsulation material and it was possible to extract a single gram per application, this would mean that 30 doses of the immunomodulator would have to be injected or disposed within the encapsulation material, i.e., the transdermal cream. To facilitate this, the dilution must be at the appropriate level. If, for example, 3 mL of diluted immunomodulator were added to the 30 g of viscous encapsulation material, then 0.3 mL of the diluted immunomodulator would constitute a single dose. In that example, suppose that the allergist or medical professional determined that this was the case and required five separate immunomodulators to be disposed within the viscous encapsulation material. This would then require the consolidator to basically add 3 mL of diluted immunomodulators from the desired vial or bottle having the desired dilution of the desired immunomodulators or antigen. For this example, this would require three injections, each being 3 mL. The actual increase in the volume of the viscous encapsulation material disposed within the container, containing 30 g of viscous encapsulation material would only be slightly greater than that value. If the distribution mechanism were able to distribute a single grand of viscous encapsulation material as a single “dose” of the material, this would actually provide a single dose of five different antigens in a single application of 1 g of the material onto the skin. In the example of FIG. 3, this would require, for each volume of eight dose of viscous encapsulation material, that the appropriate amount of immunomodulators or antigen be disposed therein.

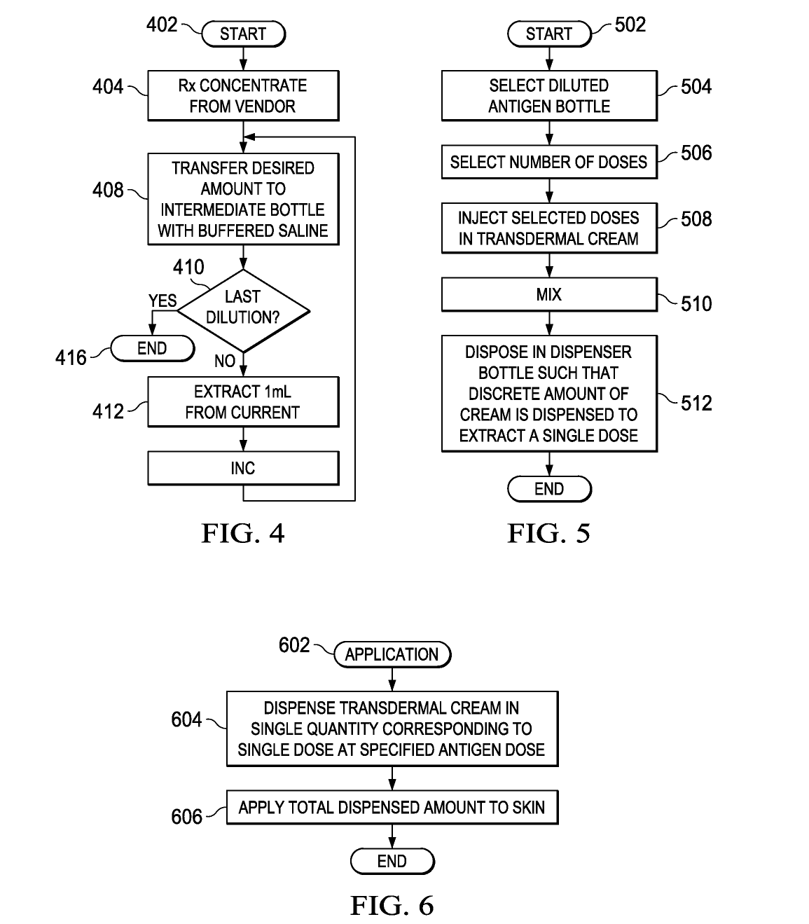

Referring now to FIG. 4, there is illustrated a process flow for the embodiment of FIG. 1. This is initiated at a process block 402 and then proceeds to block 404 wherein a certain amount of concentrated extract is received from a vendor, this being a qualified or authorized vendor for the extract. This is typically at a predetermined concentrate level of, for example, 1:20 m/v. The process then flows to a block 408 wherein a defined quantity of, for example, 0.45 mL is transferred to a 5 mL bottle which already has a quantity of 4.5 mL buffered saline solution disposed therein. The process then flows to a block 410 to determine if this was the last dilution step needed, as described hereinabove, depending upon what level of dilution is necessary. If, for example, by steps of dilution are required for a particular patient, and all five steps would be processed. However, it is not necessary to do all five steps if an intermediate dilution is required. This essentially customizes the overall operation for a particular patient. Further, the industry is so regulated such that only 5 mL bottles can be utilized for this dilution process. Thus, it will only be a maximum of 5 mL of diluted material available at any step prior to proceeding to the next step. Thus, if all 5 mL are required, then the next step is not desired or useful. If it is not the last dilution step, the process flows to a block 412 to extract 0.45 mL of diluted antigen from the current 5 mL bottle and then flows back to the input of the process block 408 after incrementing the bottle count. This continues until the last dilution, at which time the process flows from the block 410 to a terminate block 416.

Referring now to FIG. 5, there is illustrated a process flow for combining the selected diluted antigen with the viscous encapsulation material or transdermal cream, which is initiated at a block 502 and proceeds to a process block 504 to select the diluted antigen bottle or bottles from which to extract the dose or doses. At a process block 506, the prescribed number of doses is extracted from that particular bottle, keeping in mind that only a maximum of 5 mL of the diluted antigen is available. At the process block 508, the selected number of doses of the diluted antigen is injected into a predetermined quantity of transdermal cream from one bottle for a single antigen mixture and from multiple bottles for a multiple antigen mixture. The process then flows to block 510 to mix the injected cream. The process then flows to a block 512 to dispose the mixed transdermal cream in a dispenser bottle such that a discreet amount of transdermal cream can be dispensed so as to extract a single dose of the diluted antigen contained therein.

Referring now to FIG. 6, there is illustrated a process flow for the application of the transdermal cream. This is initiated at a process block 602 and then the flow proceeds to process block 604. The transdermal cream is dispensed in a single quantity that corresponds to a single dose at the specified antigen dose. This instruction is provided by a medical professional and, if followed, is substantially identical to the subcutaneous injection of the same dose directly from the hypodermic needle. Once dispensed, this amount of transdermal cream is applied to the skin by the patient or a medical professional, as indicated by a process block 606.

As an example, suppose that the container provided to the patient contained in 30 g of viscous encapsulation material or transdermal cream. In this material was contained a plurality of doses such that a single dose is contained within 1 g of material. The physician might provide a schedule or regimen that, in the first week requires one dose to be applied on Monday, Wednesday and Friday of that week. This would require the patient to dispense a single gram of material on each of those days constituting a single dose for each of those days and apply it. In the next week, the regimen is to apply two doses three times a week on Monday, Wednesday and Friday, requiring the patient to dispense 2 g of material for each of those dates. In the third week, the regimen might require three doses to be applied three times in that week on Monday, Wednesday and Friday. This requires the patient to dispense 3 g of material for each of those days and apply it. In the fourth week, the regimen might require the patient to dispose four doses of the material onto the skin on three days of the week, Monday, Wednesday and Friday. This would require the patient to dispense 4 g of material for each of those three days and apply it.

The application transdermal cream could be to any portion of the skin, but preferably, it would be to an area that represents an area of some type of allergic reaction. If for example, the patient experiences some type of rash on their neck, they would apply the mixture to that area of the body. If not, it could be applied only to the arm.

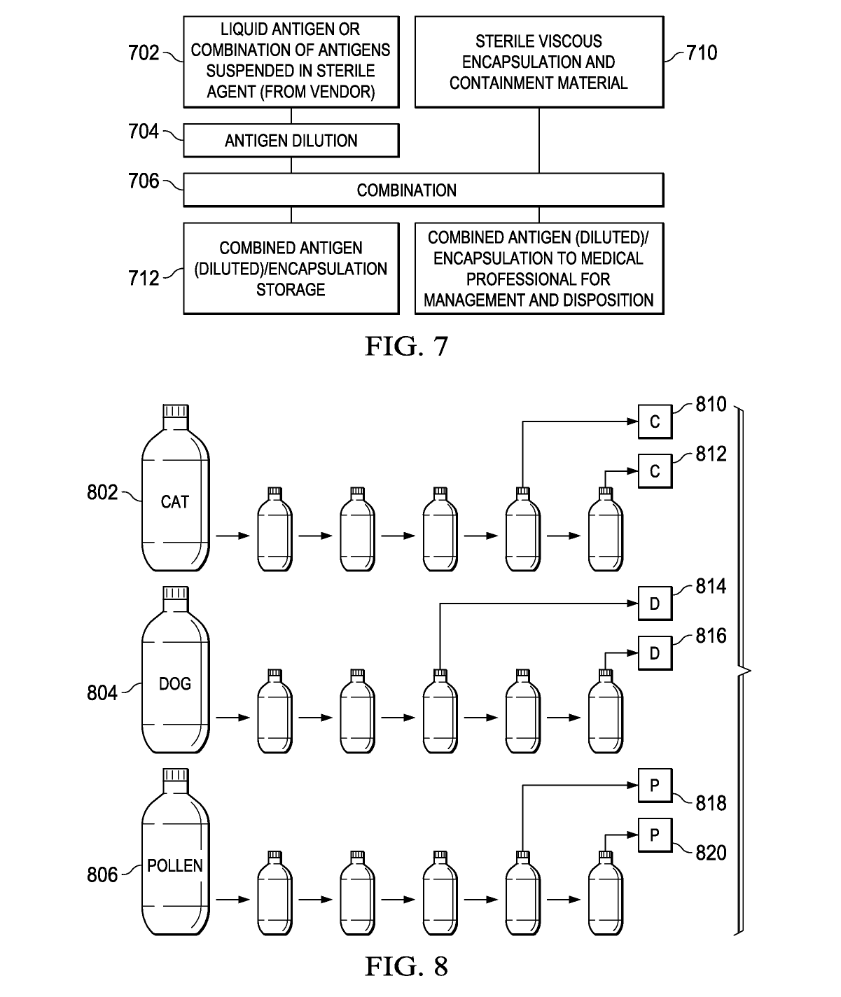

Referring now to FIG. 7, there is illustrated in overall flow of the operation of moving concentrated antigen from a vendor to an end user. As noted hereinabove, the liquid antigen in a concentrated extract was first received from a vendor, which is basically a combination of a single antigen or antigens suspended in a sterile agent. This is indicated by a block 702. The antigen is then diluted from this extract to a desired diluted level, as indicated by a process block 704. This is combined in a block 706 with a sterile viscous encapsulation and containment material, i.e., a transdermal cream, for distribution. This, as described hereinabove, will typically be a defined number of doses of a single diluted antigen or multiple diluted antigens, wherein a dose is again defined as being a typical dose that a medical professional would administer to a patient in an office visit necessary to achieve a therapeutic result. This is either transferred as a combined antigen (diluted)/encapsulation product for storage on a shelf, as indicated by a block 712, or it would be transferred to a medical professional for management and disposition.

Referring now to FIG. 8, there is illustrated a diagrammatic view of three different extracts of antigens/allergens 802, 804 and 806. Each of these is for a particular antigen or allergen. The first two are for antigens respectively associated with a cat and a dog. The third is for an allergen associated with pollen. They are each diluted in accordance with the process described hereinabove with respect to FIG. 1. As illustrated, the antigen extract in bottle 802 is transferred as a diluted level to either an encapsulation material in a container 810 or 812, each at a different diluted level. This is similarly the case with respect to the antigen in bottle 804 and the allergen in 806 wherein the diluted level of the antigen in the bottle 804 is disposed in containers 814 and 816 and the diluted level of the allergen in bottle 806 is disposed in containers 818 and 820. Typically, any extract will be 100% pure with respect to the particular extract. These concentrated extracts are not typically mixed, which is typically a function that the medical professional will perform. This, of course, is a customized mixture for a particular patient. For storage on the shelf, the operation of FIG. 8 will be facilitated in order to ensure that the containers 810-820 contained only a single antigen. Thus, when transferring the container to a store, for example, this would be stored on the shelf as a single allergen combination. This is for a situation where in a single bottle or container of the viscous encapsulation material or transdermal cream would be associated with only a single immunomodulator or antigen.

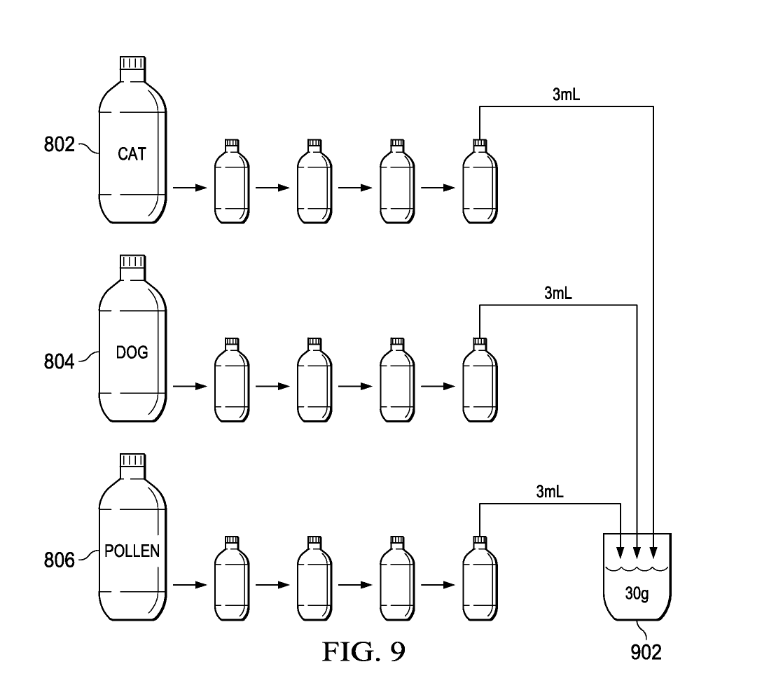

Referring now to FIG. 9, there is illustrated an alternate disclosure to that of the embodiment of FIG. 8. In this embodiment, each of the immunomodulators or antigens at the concentrated levels in the bottles 802-806 are diluted in accordance with the process noted hereinabove wherein they are sequentially diluted in the associated 5 mL bottles. However, note that only a maximum of 5 mL can be extracted from a given bottle at the last dilution level. If, in this example, it is desired to distribute a predefined number of doses to a final carrier 902 having 30 g of viscous encapsulation material or transdermal cream disposed therein and each dose will be associated with a single gram of that material, then the amount of diluted antigen must be adjusted such that single dose is contained within 0.3 mL of the material. Thereafter, if 3 mL of antigen is extracted from a given bottle, this constitutes 30 doses such that a single dose will be associated with a single gram of the final encapsulation material. In this example, from each of the last dilution bottles for each of the concentrate bottles 802-804, 3 mL in extracted and inserted within the container 902 containing 30 g of material. Thus, for each gram of material, there will be a single dose of the particular antigen associated there with. Thus, encapsulation material in the container 902 now acts as a consolidator of all of the antigens. It is noted that, if the starting material is, for example, 30 g, and there is provided a dispenser capable of dispensing a single gram of material at a time, the addition of the antigens might increase the -volume slightly. This, of course, depends upon what the volume of cream constitutes for a gram of cream for the viscous encapsulation material. This may increase the overall quantity or volume by a small percentage, but, in general, the overall volume is not increased. If, for example, a large number of antigens were introduced into the viscous material 902 in this matter, the initial volume of the viscous material magnitude could be decreased to account for such, yielding a total container having 30 g disposed therein which is comprised of the viscous encapsulation material and all of the particular antigens or immunomodulators desired.

As a distribution mechanism, all that is necessary is for the consolidator to have available a kit with the appropriate dilution bottles and the concentrated extract for each concentrated extract desired. The amount of material from one bottle to the next is then defined such that the consolidator can accurately control the amount of diluted antigen that is in the last bottle. This would then allow the consolidator to create the container 902 containing the appropriate amount of doses of antigens therein. Since a particular kit may have a single bottle of concentrated extract therein, there may be multiple dilution bottles provided to allow more than one final dilution to provide another consolidated mixture of transdermal cream with a different combination of antigens.

Allergies are the leading cause of asthma and asthma is a debilitating, life threatening, disease. It costs millions of dollars to treat. Allergy immunotherapy is the only known treatment for the prevention of asthma and further is the only treatment that may possibly reverse the disease. The topical antigen allergy treatments will likely affect 90% of allergy and asthma patients.

It will be appreciated by those skilled in the art having the benefit of this disclosure that this method provides a method for delivery of immunomodulators to a patient across the skin barrier of the patient. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. A method for delivering an immunomodulator to a patient, comprising the steps of:
providing a container of concentrated immunomodulator extract;
diluting the immunomodulator extract with a predetermined dilutant in a sterile container approved for such dilution and to a desired dilution by transferring a desired quantity of the concentrated immunomodulator to the sterile container, the sterile container having a defined volume of diluted immunomodulator after dilution thereof;
selecting a prescribed amount from the sterile container required to provide a predetermined number of doses thereof, there being at least one dose selected, a dose providing a desired therapeutic effect to a patient;
providing a viscous encapsulation material that is able to carry immunomodulators across the dermis of a patient and having a defined volume;
introducing an amount of the diluted immunomodulator comprising one or more doses of diluted immunomodulator into the viscous encapsulation material;
disposing a prescribed amount of viscous encapsulation material containing the introduced diluted antigen therein within a container that is able to dispense such viscous encapsulation material containing the introduced diluted antigen in at least a discrete amount associated with each dose contained therein;
dispensing from the container the amount of viscous encapsulation material containing the introduced diluted immunomodulator in an amount equal to at least a single dose; and
applying the dispensed amount of viscous encapsulation material containing the introduced diluted antigen to the skin by the patient or a medical professional.

2. The method of claim 1, wherein the step of diluting comprises the steps of:
providing a plurality of sterile containers, each associated with a different dilution level;
extracting a defined amount of concentrated immunomodulators extract from the container of immunomodulators extract and disposing it within a first one of the sterile containers containing a dilutant to provide a first dilution level;
extracting a defined amount of the diluted immunomodulators at the first solution from the first of the sterile containers and disposing it within the second of the sterile containers to provide a second dilution level; and
progressively extracting a defined amount of diluted immunomodulators from a previous one of the sterile containers to the next thereof containing a dilutant to provide progressively more diluted levels until the last of the sterile containers containing a final dilution level.

3. The method of claim 1, wherein the immunomodulators comprise an antigen or an allergen.

4. The method of claim 1, wherein the viscous encapsulation material comprises a transdermal cream.

5. The method of claim 1, wherein the viscous encapsulation material is operable, when applied to the stratum corneum of the patient is operable to cross the dermis of the patient to subcutaneously deliver the immunomodulators.

6. The method of claim 1, wherein the step of disposing a prescribed amount of viscous encapsulation material containing the introduced diluted antigen therein within a container comprises disposing a fixed volume of viscous encapsulation material containing the introduced diluted antigen therein within the container with a fixed amount of discrete increments, where in the container is operable to selectively output should discrete increments individually and, wherein the step of introducing an amount of the diluted immunomodulators comprises, for each diluted immunomodulators desired to be included within the viscous encapsulation material, determining the amount of each desired diluted immunomodulators constitutes a dose and, for each thereof, multiplying the dosage amount by the number of fixed discrete increments available within the viscous encapsulation material within the container and introducing such amount into the viscous encapsulation material within the container.

7. The method of claim 1, wherein the immunomodulators include antigens for treating allergies and non-antigens.

8. A method for creating a consolidated compound for delivering an immunomodulator to a patient, comprising the steps of:
providing a plurality of containers of concentrated immunomodulator extract;
for each container of concentrated immunomodulator extract, diluting the immunomodulator extract with a predetermined dilutant in an associated sterile container approved for such dilution and to a desired dilution by transferring a desired quantity of the concentrated immunomodulator to the associated sterile container, the associated sterile container having a defined volume of diluted immunomodulator after dilution thereof, such that there is an associated sterile container for each container of concentrated immunomodulator extract
providing a viscous encapsulation material that is able to carry immunomodulators across the dermis of a patient and having a defined volume disposed within a container, the defined volume divided into a plurality of dispensable increments;
selecting a prescribed amount from each of the sterile containers associated with each of the containers of concentrated immunomodulator, the prescribed amount for each of the sterile containers defined as that amount of the diluted immunomodulator extract required to provide a number of doses equal to the number of dispensable increments from the container containing the viscous encapsulation material, a dose providing a desired therapeutic effect to a patient for each of the diluted immunomodulator extracts;
introducing the selected amount of each of the diluted immunomodulator extract into the viscous encapsulation material; and
mixing the introduced amount of each of the diluted immunomodulator extracts with the viscous encapsulating material in which it was introduced.

9. The method of claim 8, wherein the step of diluting comprises the steps of:
providing a plurality of sterile containers, each associated with a different dilution level;
associated with each container of concentrated immunomodulators extract, extracting a defined amount of concentrated immunomodulator extract from the container of immunomodulator extract and disposing it within a first one of the associated sterile containers containing a dilutant to provide a first dilution level;
extracting a defined amount of the diluted immunomodulator extract at the first solution from the first of the associated sterile containers and disposing it within the second of the associated sterile containers to provide a second dilution level; and
progressively extracting a defined amount of diluted immunomodulator extract from a previous one of the associated sterile containers to the next thereof containing a dilutant to provide progressively more diluted levels until the last of the associated sterile containers containing a final dilution level.

10. The method of claim 8, wherein the immunomodulators comprise an antigen or an allergen.

11. The method of claim 8, wherein the viscous encapsulation material comprises a transdermal cream.

12. The method of claim 8, wherein the viscous encapsulation material is operable, when applied to the stratum corneum of the patient is operable to cross the dermis of the patient to subcutaneously deliver the immunomodulators.

13. A method for providing a plurality of diluted immunomodulators for the purpose of creating a consolidated compound for delivering an immunomodulator to a patient, wherein the base of the compound comprises a viscous encapsulation material that is able to carry immunomodulators across the dermis of a patient and having a defined volume disposed within a container, the defined volume divided into a plurality of dispensable increments comprising the steps of:
providing a plurality of containers of concentrated immunomodulator extract;
for each container of concentrated immunomodulator extract, diluting the immunomodulator extract with a predetermined dilutant in an associated sterile container approved for such dilution and to a desired dilution by transferring a desired quantity of the concentrated immunomodulator to the associated sterile container, the associated sterile container having a defined volume of diluted immunomodulator after dilution thereof, such that there is an associated sterile container for each container of concentrated immunomodulator extract; and
selecting a prescribed amount from each of the sterile containers associated with each of the containers of concentrated immunomodulator, the prescribed amount for each of the sterile containers defined as that amount of the diluted immunomodulator extract required to provide a number of doses equal to the number of dispensable increments from the container containing the viscous encapsulation material, a dose providing a desired therapeutic effect to a patient for each of the diluted immunomodulator extracts;
wherein there are provided a plurality of selected amounts of diluted immunomodulator extract, each selected amount corresponding to the number of doss required to equal the number of dispensable increments of viscous encapsulation material.

14. The method of claim 13, wherein the step of diluting comprises the steps of:
providing a plurality of sterile containers, each associated with a different dilution level;
associated with each container of concentrated immunomodulators extract, extracting a defined amount of concentrated immunomodulator extract from the container of immunomodulator extract and disposing it within a first one of the associated sterile containers containing a dilutant to provide a first dilution level;
extracting a defined amount of the diluted immunomodulator extract at the first solution from the first of the associated sterile containers and disposing it within the second of the associated sterile containers to provide a second dilution level; and
progressively extracting a defined amount of diluted immunomodulator extract from a previous one of the associated sterile containers to the next thereof containing a dilutant to provide progressively more diluted levels until the last of the associated sterile containers containing a final dilution level.

15. The method of claim 13, where in the immunomodulators comprise an antigen or an allergen.

16. The method of claim 13, where in the viscous encapsulation material comprises a transdermal cream.
